(19) United States
(12) Patent Application Publication
(10) Pub. No.: US 2021/0304868 A1
(43) Pub. Date: Sep. 30, 2021

(54) ARTIFICIAL INTELLIGENCE METHODS AND SYSTEMS FOR MULTI-FACTOR SELECTION PROCESS

(71) Applicant: KPN Innovations, LLC, Lakewood, CO (US)

(72) Inventor: Kenneth Neumann, Lakewood, CO (US)

(21) Appl. No.: 16/836,008

(22) Filed: Mar. 31, 2020

(51) Int. Cl. G16H 20/60; G06N 20/00

(52) U.S. Cl. CPC G16H 20/60; G06N 20/00

(57) ABSTRACT

An artificial intelligence system for multi-factor selection process, the system comprising a computing device, the computing device designed and configured to receive an equivalency request, retrieve at least an element of user data, determine a nutritional output utilizing the equivalency request and the at least an element of user data, and calculate an optimization value utilizing the nutritional output.

### FIELD OF THE INVENTION

The present invention generally relates to the field of artificial intelligence. In particular, the present invention is directed to artificial intelligence methods and systems for multi-factor selection process.

### BACKGROUND

Efficient calculations of allotments can be challenging. Often, there are a multitude of factors to consider, which may lead to varying results. A lack of uniformity may end in dissatisfaction with varying resulting options.

### SUMMARY OF THE DISCLOSURE

In an aspect, an artificial intelligence system for multi-factor selection process, the system comprising a computing device, the computing device designed and configured to receive an equivalency request, wherein the equivalency request contains a user specified individualized level, retrieve at least an element of user data and determine a user metabolic state utilizing the at least a retrieved element of user data and a classification algorithm, determine a nutritional output utilizing the equivalency request, the user metabolic state, the at least an element of user data, and at least a machine-learning process, and calculate an optimization value utilizing the nutritional output.

In an aspect, an artificial intelligence method of multi-factor selection process, the method comprising receiving by a computing device an equivalency request, wherein the equivalency request contains a user specified individualized level. The method includes retrieving by the computing device at least an element of user data and determining a user metabolic state utilizing the at least a retrieved element of user data and a classification algorithm. The method includes determining by the computing device a nutritional output utilizing the equivalency request and the at least an element of user data. The method includes calculating by the computing device an optimization value utilizing the nutritional output.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of an artificial intelligence system for multi-factor selection process.

FIG. 2 is a block diagram illustrating an exemplary embodiment of a user database.

FIG. 3 is a block diagram illustrating an exemplary embodiment of a nutritional database.

FIG. 4 is a block diagram illustrating an exemplary embodiment of a process database.

FIG. 5 is a process flow diagram illustrating an exemplary embodiment of an artificial intelligence method of multi-factor selection process; and

FIG. 6 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to artificial intelligence systems and methods for multi-factor selection process. In an embodiment, machine-learning is utilized to determine a nutritional output. A biological extraction and/or user entered information regarding a user's health history is utilized to optimize the output. A nutritional output is utilized in combination with other factors to calculate an optimization value that contains a total allotment.

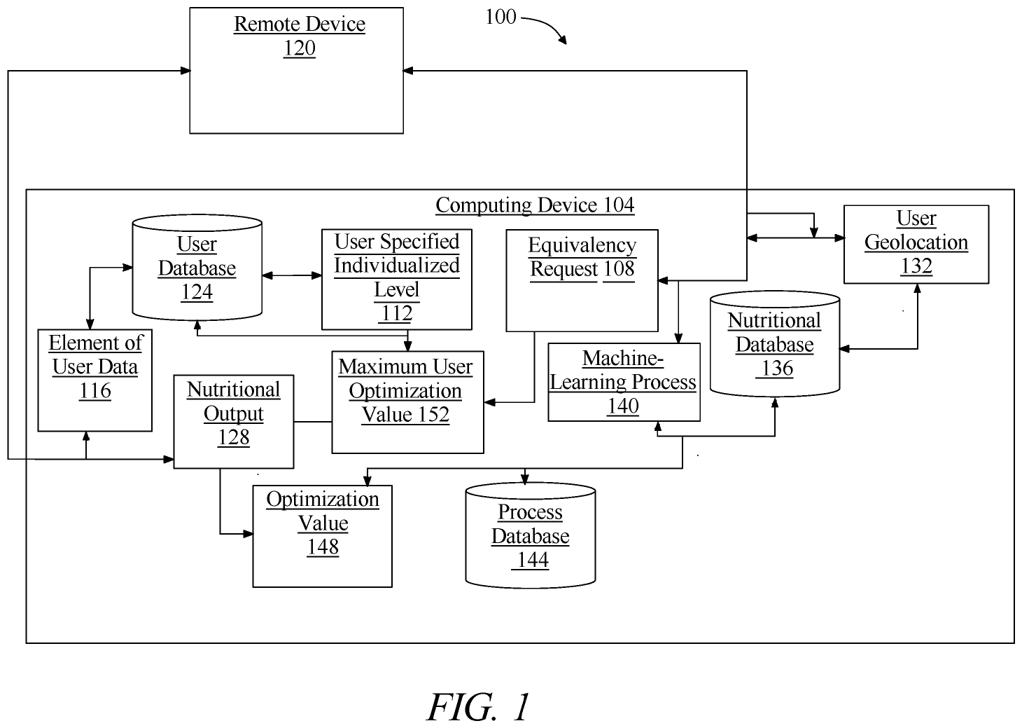

Referring now to FIG. 1, an exemplary embodiment of an artificial intelligence system 100 for multi-factor selection process is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices 104 in a first location and a second computing device 104 or cluster of computing devices 104 in a second location. Computing device 104 may include one or more computing devices 104 dedicated to data storage, security, distribution of traffic for load balancing, and the like. Computing device 104 may distribute one or more computing tasks as described below across a plurality of computing devices 104 of computing device 104, which may operate in parallel, in series, redundantly, or in any other manner used for distribution of tasks or memory between computing devices 104. Computing device 104 may be implemented using a “shared nothing” architecture in which data is cached at the worker, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive an equivalency request 108. An “equivalency request,” as used in this disclosure, is data indicating a particular level of how personalized and how precise a user seeks to have nutrition optimized to the user's body. An equivalency request 108 may specify how well a user wants nutrition and/or meals matched to the personal nutritional requirements of a user's body. “Nutrition,” as used in this disclosure, is any substance suitable for consumption by a human being. Nutrition may include an ingredient such as a banana or a stalk of celery. Nutrition may include an animal product such as lamb, a protein such as tofu, an herb such as rosemary, a spice such as cinnamon and the like. Nutrition may include a meal such as a dinner consisting of spaghetti and meatball or a snack consisting of almonds and cheddar cheese. Nutrition may include one or more options available to order on a menu such as a dish of fish and chips that may be ordered from a seafood restaurant or an acai bowl from a smoothie shop. Nutrition may include supplements, which may include any product intended to supplement a user's diet. Supplements may include products consumed by a user that contain a dietary ingredient. Dietary ingredients may include any vitamin, mineral, nutrient, homeopathic, amino acid, herb, botanical, nutraceutical, enzyme, health food, medical food, and the like. Supplements may contain dietary ingredients sourced from food, synthesized in a laboratory, and/or sourced in combination. Supplements may include for example, a multi-vitamin, co-enzyme q10, ubiquinol, resveratrol, probiotics such as Lactobacillus Acidophilus, Bifidobacterium Bifidum, Saccharomyces Boulardii, fish oil, B-Vitamin complex, Vitamin D, cranberry, products containing combination ingredients, and the like. Supplements may be available in a variety of different dosage forms for a user to consume including for example, capsules, tablets, pills, buccal tablets, sub-lingual tablets, orally-disintegrating products, thin films, liquid solution, liquid suspension, oil suspension, powder, solid crystals, seeds, foods, pastes, buccal films, inhaled forms such as aerosols, nebulizers, smoked forms, vaporized form, intradermal forms, subcutaneous forms, intramuscular forms, intraosseous forms, intraperitoneal forms, intravenous forms, creams, gels, balms, lotion, ointment, ear drops, eye drops, skin patch, transdermal forms, vaginal rings, dermal patch, vaginal suppository, rectal suppository, urethral suppository, nasal suppository, and the like. Supplements may be available to a user without a prescription such as for example, a fish oil supplement sold at a health food store. Supplements may be available to a user with a prescription, such as for example subcutaneous cyanocobalamin injections available at a compounding pharmacy. Supplements may be categorized into different grade products such as for example pharmaceutical grade supplements that may contain in excess of 99% purity and do not contain binders, fillers, excipients, dyes, or unknown substances and are manufactured in Food and Drug Administration (FDA) registered facilities that follow certified good manufacturing practices (cGMP); supplements may be of food grade quality such as for example supplements deemed to be suitable for human consumption; supplements may be of feed grade quality such as for example supplements deemed to be suitable for animal consumption.

With continued reference to FIG. 1, an equivalency request 108 contains a user specified individualized level 112. A “user specified individualized level,” as used in this disclosure, is any data, including any numerical and/or character describing a user's preference scaled on a continuum, as to how personalized and/or customized the user prefers the user's nutrition. A user specified individualized level 112 may indicate on a continuum, such as an indication of “low” when a user is not concerned about nutrition customized to the user's personal nutritional needs. A user specified individualized level 112 may indicate “high” when a user is concerned about nutrition and seeks to have nutrition highly optimized based on the user's nutritional needs. A user specified individualized level 112 may indicate “medium” when a user is moderately concerned about nutrition customized to the user's personal nutrition needs.

With continued reference to FIG. 1, computing device 104 is configured to retrieve at least an element of user data 116. An “element of user data,” as used in this disclosure, is any medical and/or health history data pertaining to a user. An element of user data 116 may include a user reported element of user data 116. A user reported element of user data 116 may include any medical data pertaining to a user, supplied by a user. For example, a user reported element of user data 116 may include any previous health history, health records, diagnosis, medications, treatments, major surgeries, complications, and the like that the user may be suffering from. For example, a user reported element of user data 116 may include an anaphylactic reaction to all tree nuts that the user was diagnosed with as a young child. In yet another non-limiting example, a user reported element of user data 116 may describe a previous diagnosis such as endometriosis that the user was diagnosed with three years back, and treatments that the user engages in to manage her endometriosis, including supplementation with fish oil and following a gluten free diet. In yet another non-limiting example, a user may provide one or more elements of health history information, such as when a user may select how much of a user's medical records the user seeks to share with computing device 104. For example, a user may prefer to share only the user's hospitalization records and not the user's current medication list. In yet another non-limiting example, a user may seek to share as many records as are available for the user, such as the user's entire vaccination history. In yet another non-limiting example, a user may share health history information that is available to the user, such as when records may become lost or misplaced. In yet another non-limiting example, an element of user data may contain one or more readings obtained from a computing device 104 connected to a directly operating sensor. A sensor may contain any sensor as described herein. Sensor may obtain one or more readings pertaining to a user's body, including one or more bio-physiological signals including but not limited to respiration rate, electrocardiography, electromyography, blood volume pressure, blood pressure, electrooculography, pulse rate, muscle activity, brain activity skin hydration, heart rate, inter-beat-interval, heart rate variability, eye pupil size, pupil movement, depth of breath and the like. In yet another non-limiting example, a sensor may include any sensor worn on a user's body, such as a sensor embedded headband that may measure one or more markers of sleep, a sensor embedded in a user's mouth that may measure one or more nutrient level obtained from food that a user consumes, or a sensor that may be embedded under a user's skin such as in a microchip that may measure one or more bio-physiological signals and/or nutrient levels of a user.

With continued reference to FIG. 1, computing device 104 determines a user metabolic state utilizing at least a retrieved element of user data and a classification algorithm. A “user metabolic state,” as used in this disclosure, is any indication as to the way in which a user processes food to be used for energy and/or growth. A user metabolic state may indicate how quickly a user burns calories. A user metabolic state may indicate how efficiently a user converts food and nutrition into energy through organic and chemical processes. For example, a user metabolic state may indicate that a user with impaired fasting glucose does not efficiently convert carbohydrates into energy. In yet another non-limiting example, a user metabolic state may indicate that a user who is very active and participates in various exercise routines and exercise protocols is hyper-efficient at converting food into energy. In yet another non-limiting example, a user metabolic state may indicate that a non-active user who is obese may not efficiently convert food into efficient sources of energy.

With continued reference to FIG. 1, computing device 104 determines a user metabolic state utilizing a classification algorithm. A “classification algorithm,” as used in this disclosure, is a process whereby a computing device 104 derives, from training data, a model for sorting inputs into categories or bins of data. Training data includes any of the training data as described herein. Classification may be performed using, without limitation, linear classifiers such as without limitation logistic regression and/or naive Bayes classifiers, nearest neighbor classifiers including without limitation k-nearest neighbors classifiers, support vector machines, decision trees, boosted trees, random forest classifiers, and/or neural network-based classifiers.

With continued reference to FIG. 1, classification algorithm may include generating a Naïve Bayes classification algorithm. Naïve Bayes classification algorithm generates classifiers by assigning class labels to problem instances, represented as vectors of feature values. Class labels are drawn from a finite set. Naïve Bayes classification algorithm may include generating a family of algorithms that assume that the value of a particular feature is independent of the value of any other feature, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming classification training data into a frequency table. Computing device 104 may then calculate a likelihood table by calculating probabilities of different data entries and classification labels. Computing device 104 utilizes a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when feature vectors are binary. Naïve Bayes classification algorithm utilizes training data and at least a retrieved element of user data as an input to output a user metabolic state. A metabolic state may be identified utilizing a classification label, where a “classification label” as used in this disclosure, includes a label that indicates whether an input belongs to a particular class or not. In an embodiment, a classification label may include an indication as to the metabolic state of the user. For example, a user with hyperthyroidism who is a hyper-metabolizer may be classified to a metabolic state that indicates that the user is a hypermetabolizer, whereas a user who is not active, and does not engage in physical activity may be classified to a metabolic state that indicates that the user is a slow metabolizer.

With continued reference to FIG. 1, classification algorithm may include generating a K-nearest neighbor (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute 1 as derived using a Pythagorean norm: l=\sqrt{Eᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference to FIG. 1, computing device 104 may receive a user reported element of user data 116 from an input generated by a user utilizing a remote device 120. A remote device 120 may include without limitation, a display in communication with computing device 104, where a display may include any display as described herein. Remote device 120 may include an additional computing device, such as a mobile device, laptop, desktop, computer and the like.

With continued reference to FIG. 1, an element of user data 116 may include a biological extraction. A “biological extraction,” as used in this disclosure, is an element of data including at least an element of user physiological data. As used in this disclosure, “physiological data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, physiological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss.

With continued reference to FIG. 1, physiological state data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological state data may include, without limitation, immune function data such as Interleukine-6 (IL-6), TNF-alpha, systemic inflammatory cytokines, and the like.

Continuing to refer to FIG. 1, physiological state data may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological state data may include measures of glucose metabolism such as fasting glucose levels and/or hemoglobin A1-C(HbA1c) levels. Physiological state data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological state data may include measures of estimated glomerular filtration rate (eGFR). Physiological state data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological state data may include antinuclear antibody levels. Physiological state data may include aluminum levels. Physiological state data may include arsenic levels. Physiological state data may include levels of fibrinogen, plasma cystatin C, and/or brain natriuretic peptide.

Continuing to refer to FIG. 1, physiological state data may include measures of lung function such as forced expiratory volume, one second (FEV-1) which measures how much air can be exhaled in one second following a deep inhalation, forced vital capacity (FVC), which measures the volume of air that may be contained in the lungs. Physiological state data may include a measurement blood pressure, including without limitation systolic and diastolic blood pressure. Physiological state data may include a measure of waist circumference. Physiological state data may include body mass index (BMI). Physiological state data may include one or more measures of bone mass and/or density such as dual-energy x-ray absorptiometry. Physiological state data may include one or more measures of muscle mass. Physiological state data may include one or more measures of physical capability such as without limitation measures of grip strength, evaluations of standing balance, evaluations of gait speed, pegboard tests, timed up and go tests, and/or chair rising tests.

Still viewing FIG. 1, physiological state data may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological state data may include one or more evaluations of sensory ability, including measures of audition, vision, olfaction, gustation, vestibular function and pain.

Continuing to refer to FIG. 1, physiological state data may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices 104; for instance, user patterns of purchases, including electronic purchases, communication such as via chat-rooms or the like, any textual, image, video, and/or data produced by the subject, any textual image, video and/or other data depicting and/or describing the subject, or the like. Any psychological data and/or data used to generate psychological data may be analyzed using machine-learning and/or language processing module as described in this disclosure. As a non-limiting example, biological extraction may include a psychological profile; the psychological profile may be obtained utilizing a questionnaire performed by the user.

Still referring to FIG. 1, physiological state data may include genomic data, including deoxyribonucleic acid (DNA) samples and/or sequences, such as without limitation DNA sequences or other genetic sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological state data may include proteomic data, which as used herein is data describing all proteins produced and/or modified by an organism, colony of organisms, or system of organisms, and/or a subset thereof. Physiological state data may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological state data of a person, as described in further detail below.

With continuing reference to FIG. 1, physiological state data may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals or the like, or any other user-entered data that a user may provide to a medical professional when seeking treatment and/or evaluation, and/or in response to medical intake papers, questionnaires, questions from medical professionals, or the like. Physiological state data may include any physiological state data, as described above, describing any multicellular organism living in or on a person including any parasitic and/or symbiotic organisms living in or on the persons; non-limiting examples may include mites, nematodes, flatworms, or the like. Examples of physiological state data described in this disclosure are presented for illustrative purposes only and are not meant to be exhaustive.

With continued reference to FIG. 1, physiological data may include, without limitation any result of any medical test, physiological assessment, cognitive assessment, psychological assessment, or the like. System 100 may receive at least a physiological data from one or more other devices after performance; system 100 may alternatively or additionally perform one or more assessments and/or tests to obtain at least a physiological data, and/or one or more portions thereof, on system 100. For instance, at least physiological data may include or more entries by a user in a form or similar graphical user interface object; one or more entries may include, without limitation, user responses to questions on a psychological, behavioral, personality, or cognitive test. For instance, at least a server may present to user a set of assessment questions designed or intended to evaluate a current state of mind of the user, a current psychological state of the user, a personality trait of the user, or the like; at least a server may provide user-entered responses to such questions directly as at least a physiological data and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 1, assessment and/or self-assessment data, and/or automated or other assessment results, obtained from a third-party device 116; third-party device 116 may include, without limitation, a server or other device (not shown) that performs automated cognitive, psychological, behavioral, personality, or other assessments. Third-party device 116 may include a device operated by an informed advisor. An informed advisor may include any medical professional who may assist and/or participate in the medical treatment of a user. An informed advisor may include a medical doctor, nurse, physician assistant, pharmacist, yoga instructor, nutritionist, spiritual healer, meditation teacher, fitness coach, health coach, life coach, and the like.

With continued reference to FIG. 1, physiological data may include data describing one or more test results, including results of mobility tests, stress tests, dexterity tests, endocrinal tests, genetic tests, and/or electromyographic tests, biopsies, radiological tests, genetic tests, and/or sensory tests. Persons skilled in the art, upon reviewing the entirety of this disclosure, will be aware of various additional examples of at least a physiological sample consistent with this disclosure.

With continued reference to FIG. 1, physiological data may include one or more user body measurements. A “user body measurement” as used in this disclosure, includes a measurable indicator of the severity, absence, and/or presence of a disease state. A “disease state” as used in this disclosure, includes any harmful deviation from the normal structural and/or function state of a human being. A disease state may include any medical condition and may be associated with specific symptoms and signs. A disease state may be classified into different types including infectious diseases, deficiency diseases, hereditary diseases, and/or physiological diseases. For instance and without limitation, internal dysfunction of the immune system may produce a variety of different diseases including immunodeficiency, hypersensitivity, allergies, and/or autoimmune disorders.

With continued reference to FIG. 1, user body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

With continued reference to FIG. 1, epigenetic, as used herein, includes any user body measurements describing changes to a genome that do not involve corresponding changes in nucleotide sequence. Epigenetic body measurement may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic body measurements may include data describing one or more states of methylation of genetic material.

With continued reference to FIG. 1, gut-wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 1, gut-wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut-wall includes muscular layer which contains smooth muscle that aids in peristalsis and the movement of digested material out of and along the gut. Gut-wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 1, gut-wall body measurement may include data describing one or more test results including results of gut-wall function, gut-wall integrity, gut-wall strength, gut-wall absorption, gut-wall permeability, intestinal absorption, gut-wall barrier function, gut-wall absorption of bacteria, gut-wall malabsorption, gut-wall gastrointestinal imbalances and the like.

With continued reference to FIG. 1, gut-wall body measurement may include any data describing blood test results of creatinine levels, lactulose levels, zonulin levels, and mannitol levels. Gut-wall body measurement may include blood test results of specific gut-wall body measurements including d-lactate, endotoxin lipopolysaccharide (LPS) Gut-wall body measurement may include data breath tests measuring lactulose, hydrogen, methane, lactose, and the like. Gut-wall body measurement may include blood test results describing blood chemistry levels of albumin, bilirubin, complete blood count, electrolytes, minerals, sodium, potassium, calcium, glucose, blood clotting factors,

With continued reference to FIG. 1, gut-wall body measurement may include one or more stool test results describing presence or absence of parasites, firmicutes, Bacteroidetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 1, gut-wall body measurement may include one or more stool test results describing presence, absence, and/or measurement of microorganisms including bacteria, archaea, fungi, protozoa, algae, viruses, parasites, worms, and the like. Stool test results may contain species such as Bifidobacterium species, campylobacter species, clostridium difficile, cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 1, gut-wall body measurement may include one or more microscopic ova exam results, microscopic parasite exam results, protozoan polymerase chain reaction test results and the like. Gut-wall body measurement may include enzyme-linked immunosorbent assay (ELISA) test results describing immunoglobulin G (Ig G) food antibody results, immunoglobulin E (Ig E) food antibody results, Ig E mold results, IgG spice and herb results. Gut-wall body measurement may include measurements of calprotectin, eosinophil protein x (EPX), stool weight, pancreatic elastase, total urine volume, blood creatinine levels, blood lactulose levels, blood mannitol levels.

With continued reference to FIG. 1, gut-wall body measurement may include one or more elements of data describing one or more procedures examining gut including for example colonoscopy, endoscopy, large and small molecule challenge and subsequent urinary recovery using large molecules such as lactulose, polyethylene glycol-3350, and small molecules such as mannitol, L-rhamnose, polyethyleneglycol-400. Gut-wall body measurement may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 1, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithies' and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, microbiome body measurement may include one or more stool test results describing presence, absence, and/or measurement of microorganisms including bacteria, archaea, fungi, protozoa, algae, viruses, parasites, worms, and the like. Stool test results may contain species such as Ackerman's muciniphila, Anaerotruncus colihominis, bacteriology, Bacteroides vulgates', Bacteroides-Prevotella, Barnesiella species, Bifidobacterium longarm, Bifidobacterium species, Butyrivbrio crossotus, Clostridium species, Collinsella aerofaciens, fecal color, fecal consistency, Coprococcus eutactus, Desulfovibrio piger, Escherichia coli, Faecalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 1, microbiome body measurement may include one or more stool tests results that identify all microorganisms living a user's gut including bacteria, viruses, archaea, yeast, fungi, parasites, and bacteriophages. Microbiome body measurement may include DNA and RNA sequences from live microorganisms that may impact a user's health. Microbiome body measurement may include high resolution of both species and strains of all microorganisms. Microbiome body measurement may include data describing current microbe activity. Microbiome body measurement may include expression of levels of active microbial gene functions. Microbiome body measurement may include descriptions of sources of disease-causing microorganisms, such as viruses found in the gastrointestinal tract such as raspberry bushy swarf virus from consuming contaminated raspberries or Pepino mosaic virus from consuming contaminated tomatoes.

With continued reference to FIG. 1, microbiome body measurement may include one or more blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 1, microbiome body measurement may include one or more breath test results that identify certain strains of microorganisms that may be present in certain areas of a user's body. This may include for example, lactose intolerance breath tests, methane-based breath tests, hydrogen-based breath tests, fructose-based breath tests, Helicobacter pylori breath test, fructose intolerance breath test, bacterial overgrowth syndrome breath tests and the like.

With continued reference to FIG. 1, microbiome body measurement may include one or more urinary analysis results for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

With continued reference to FIG. 1, nutrient as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron.

With continued reference to FIG. 1, nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 1, nutrient body measurement may include one or more blood test results that identify extracellular and intracellular levels of nutrients. Nutrient body measurement may include blood test results that identify serum, white blood cell, and red blood cell levels of nutrients. For example, nutrient body measurement may include serum, white blood cell, and red blood cell levels of micronutrients such as Vitamin A, Vitamin B1, Vitamin B2, Vitamin B3, Vitamin B6, Vitamin B12, Vitamin B5, Vitamin C, Vitamin D, Vitamin E, Vitamin K1, Vitamin K2, and folate.

With continued reference to FIG. 1, nutrient body measurement may include one or more blood test results that identify serum, white blood cell and red blood cell levels of nutrients such as calcium, manganese, zinc, copper, chromium, iron, magnesium, copper to zinc ratio, choline, inositol, carnitine, methylmalonic acid (MMA), sodium, potassium, asparagine, glutamine, serine, coenzyme q10, cysteine, alpha lipoic acid, glutathione, selenium, eicosapentaenoic acid (EPA), docosahexaenoic acid (DHA), docosapentaenoic acid (DPA), total omega-3, lauric acid, arachidonic acid, oleic acid, total omega 6, and omega 3 index.

With continued reference to FIG. 1, nutrient body measurement may include one or more salivary test results that identify levels of nutrients including any of the nutrients as described herein. Nutrient body measurement may include hair analysis of levels of nutrients including any of the nutrients as described herein.

With continued reference to FIG. 1, genetic as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 1, genetic body measurement may include one or more results from one or more blood tests, hair tests, skin tests, urine, amniotic fluid, buccal swabs and/or tissue test to identify a user's particular sequence of nucleotides, genes, chromosomes, and/or proteins. Genetic body measurement may include tests that example genetic changes that may lead to genetic disorders. Genetic body measurement may detect genetic changes such as deletion of genetic material or pieces of chromosomes that may cause Duchenne Muscular Dystrophy. Genetic body measurement may detect genetic changes such as insertion of genetic material into DNA or a gene such as the BRCA1 gene that is associated with an increased risk of breast and ovarian cancer due to insertion of 2 extra nucleotides. Genetic body measurement may include a genetic change such as a genetic substitution from a piece of genetic material that replaces another as seen with sickle cell anemia where one nucleotide is substituted for another. Genetic body measurement may detect a genetic change such as a duplication when extra genetic material is duplicated one or more times within a person's genome such as with Charcot-Marie Tooth disease type 1. Genetic body measurement may include a genetic change such as an amplification when there is more than a normal number of copies of a gene in a cell such as HER2 amplification in cancer cells. Genetic body measurement may include a genetic change such as a chromosomal translocation when pieces of chromosomes break off and reattach to another chromosome such as with the BCR-ABL1 gene sequence that is formed when pieces of chromosome 9 and chromosome 22 break off and switch places. Genetic body measurement may include a genetic change such as an inversion when one chromosome experiences two breaks and the middle piece is flipped or inverted before reattaching. Genetic body measurement may include a repeat such as when regions of DNA contain a sequence of nucleotides that repeat a number of times such as for example in Huntington's disease or Fragile X syndrome. Genetic body measurement may include a genetic change such as a trisomy when there are three chromosomes instead of the usual pair as seen with Down syndrome with a trisomy of chromosome 21, Edwards syndrome with a trisomy at chromosome 18 or Patau syndrome with a trisomy at chromosome 13. Genetic body measurement may include a genetic change such as monosomy such as when there is an absence of a chromosome instead of a pair, such as in Turner syndrome.

With continued reference to FIG. 1, genetic body measurement may include an analysis of COMT gene that is responsible for producing enzymes that metabolize neurotransmitters. Genetic body measurement may include an analysis of DRD2 gene that produces dopamine receptors in the brain. Genetic body measurement may include an analysis of ADRA2B gene that produces receptors for noradrenaline. Genetic body measurement may include an analysis of 5-HTTLPR gene that produces receptors for serotonin. Genetic body measurement may include an analysis of BDNF gene that produces brain derived neurotrophic factor. Genetic body measurement may include an analysis of 9p21 gene that is associated with cardiovascular disease risk. Genetic body measurement may include an analysis of APOE gene that is involved in the transportation of blood lipids such as cholesterol. Genetic body measurement may include an analysis of NOS3 gene that is involved in producing enzymes involved in regulating vasodilation and vasoconstriction of blood vessels.

With continued reference to FIG. 1, genetic body measurement may include ACE gene that is involved in producing enzymes that regulate blood pressure. Genetic body measurement may include SLCO1B1 gene that directs pharmaceutical compounds such as statins into cells. Genetic body measurement may include FUT2 gene that produces enzymes that aid in absorption of Vitamin B12 from digestive tract. Genetic body measurement may include MTHFR gene that is responsible for producing enzymes that aid in metabolism and utilization of Vitamin B9 or folate. Genetic body measurement may include SHMT1 gene that aids in production and utilization of Vitamin B9 or folate. Genetic body measurement may include MTRR gene that produces enzymes that aid in metabolism and utilization of Vitamin B12. Genetic body measurement may include MTR gene that produces enzymes that aid in metabolism and utilization of Vitamin B12. Genetic body measurement may include FTO gene that aids in feelings of satiety or fulness after eating. Genetic body measurement may include MC4R gene that aids in producing hunger cues and hunger triggers. Genetic body measurement may include APOA2 gene that directs body to produce ApoA2 thereby affecting absorption of saturated fats. Genetic body measurement may include UCP1 gene that aids in controlling metabolic rate and thermoregulation of body. Genetic body measurement may include TCF7L2 gene that regulates insulin secretion. Genetic body measurement may include AMY1 gene that aids in digestion of starchy foods. Genetic body measurement may include MCM6 gene that controls production of lactase enzyme that aids in digesting lactose found in dairy products. Genetic body measurement may include BCMO1 gene that aids in producing enzymes that aid in metabolism and activation of Vitamin A. Genetic body measurement may include SLC23A1 gene that produce and transport Vitamin C. Genetic body measurement may include CYP2R1 gene that produce enzymes involved in production and activation of Vitamin D. Genetic body measurement may include GC gene that produce and transport Vitamin D. Genetic body measurement may include CYP1A2 gene that aid in metabolism and elimination of caffeine. Genetic body measurement may include CYP17A1 gene that produce enzymes that convert progesterone into androgens such as androstenedione, androstendiol, dehydroepiandrosterone, and testosterone.

With continued reference to FIG. 1, genetic body measurement may include CYP19A1 gene that produce enzymes that convert androgens such as androstenedione and testosterone into estrogens including estradiol and estrone. Genetic body measurement may include SRD5A2 gene that aids in production of enzymes that convert testosterone into dihydrotestosterone. Genetic body measurement may include UFT2B17 gene that produces enzymes that metabolize testosterone and dihydrotestosterone. Genetic body measurement may include CYP1A1 gene that produces enzymes that metabolize estrogens into 2 hydroxy-estrogen. Genetic body measurement may include CYP1B1 gene that produces enzymes that metabolize estrogens into 4 hydroxy-estrogen. Genetic body measurement may include CYP3A4 gene that produces enzymes that metabolize estrogen into 16 hydroxy-estrogen. Genetic body measurement may include COMT gene that produces enzymes that metabolize 2 hydroxy-estrogen and 4 hydroxy-estrogen into methoxy estrogen. Genetic body measurement may include GSTT1 gene that produces enzymes that eliminate toxic by-products generated from metabolism of estrogens. Genetic body measurement may include GSTM1 gene that produces enzymes responsible for eliminating harmful by-products generated from metabolism of estrogens. Genetic body measurement may include GSTP1 gene that produces enzymes that eliminate harmful by-products generated from metabolism of estrogens. Genetic body measurement may include SOD2 gene that produces enzymes that eliminate oxidant by-products generated from metabolism of estrogens.

With continued reference to FIG. 1, metabolic, as used herein, includes any process that converts food and nutrition into energy. Metabolic may include biochemical processes that occur within the body. Metabolic body measurement may include blood tests, hair tests, skin tests, amniotic fluid, buccal swabs and/or tissue test to identify a user's metabolism. Metabolic body measurement may include blood tests that examine glucose levels, electrolytes, fluid balance, kidney function, and liver function. Metabolic body measurement may include blood tests that examine calcium levels, albumin, total protein, chloride levels, sodium levels, potassium levels, carbon dioxide levels, bicarbonate levels, blood urea nitrogen, creatinine, alkaline phosphatase, alanine amino transferase, aspartate amino transferase, bilirubin, and the like.

With continued reference to FIG. 1, metabolic body measurement may include one or more blood, saliva, hair, urine, skin, and/or buccal swabs that examine levels of hormones within the body such as 11-hydroxy-androstereone, 11-hydroxy-etiocholanolone, 11-keto-androsterone, 11-keto-etiocholanolone, 16 alpha-hydroxyestrone, 2-hydroxyestrone, 4-hydroxyestrone, 4-methoxyestrone, androstanediol, androsterone, creatinine, DHEA, estradiol, estriol, estrone, etiocholanolone, pregnanediol, pregnanestriol, specific gravity, testosterone, tetrahydrocortisol, tetrahydrocrotisone, tetrahydrodeoxycortisol, allo-tetrahydrocortisol.

With continued reference to FIG. 1, metabolic body measurement may include one or more metabolic rate test results such as breath tests that may analyze a user's resting metabolic rate or number of calories that a user's body burns each day rest. Metabolic body measurement may include one or more vital signs including blood pressure, breathing rate, pulse rate, temperature, and the like. Metabolic body measurement may include blood tests such as a lipid panel such as low density lipoprotein (LDL), high density lipoprotein (HDL), triglycerides, total cholesterol, ratios of lipid levels such as total cholesterol to HDL ratio, insulin sensitivity test, fasting glucose test, Hemoglobin A1C test, adipokines such as leptin and adiponectin, neuropeptides such as ghrelin, pro-inflammatory cytokines such as interleukin 6 or tumor necrosis factor alpha, anti-inflammatory cytokines such as interleukin 10, markers of antioxidant status such as oxidized low-density lipoprotein, uric acid, paraoxonase 1. Persons skilled in the art, upon reviewing the entirety of this disclosure, will be aware of various additional examples of physiological state data that may be used consistently with descriptions of systems and methods as provided in this disclosure.

With continued reference to FIG. 1, physiological data may be obtained from a physically extracted sample. A “physical sample” as used in this example, may include any sample obtained from a human body of a user. A physical sample may be obtained from a bodily fluid and/or tissue analysis such as a blood sample, tissue, sample, buccal swab, mucous sample, stool sample, hair sample, fingernail sample and the like. A physical sample may be obtained from a device in contact with a human body of a user such as a microchip embedded in a user's skin, a sensor in contact with a user's skin, a sensor located on a user's tooth, and the like. Physiological data may be obtained from a physically extracted sample. A physical sample may include a signal from a sensor configured to detect physiological data of a user and record physiological data as a function of the signal. A sensor may include any medical sensor and/or medical device configured to capture sensor data concerning a patient, including any scanning, radiological and/or imaging device such as without limitation x-ray equipment, computer assisted tomography (CAT) scan equipment, positron emission tomography (PET) scan equipment, any form of magnetic resonance imagery (MM) equipment, ultrasound equipment, optical scanning equipment such as photo-plethysmographic equipment, or the like. A sensor may include any electromagnetic sensor, including without limitation electroencephalographic sensors, magnetoencephalographic sensors, electrocardiographic sensors, electromyographic sensors, or the like. A sensor may include a temperature sensor. A sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. At least a wearable and/or mobile device sensor may detect heart rate or the like. A sensor may detect any hematological parameter including blood oxygen level, pulse rate, heart rate, pulse rhythm, blood sugar, and/or blood pressure. A sensor may be configured to detect internal and/or external biomarkers and/or readings. A sensor may be a part of system 100 or may be a separate device in communication with system 100. User data may include a profile, such as a psychological profile, generated using previous item selections by the user; profile may include, without limitation, a set of actions and/or navigational actions performed as described in further detail below, which may be combined with biological extraction data and/or other user data for processes such as classification to user sets as described in further detail below.

Still referring to FIG. 1, retrieval of biological extraction may include, without limitation, reception of biological extraction from another computing device 104 such as a device operated by a medical and/or diagnostic professional and/or entity, a user client device, and/or any device suitable for use as a third-party device as described in further detail below. Biological extraction may be received via a questionnaire posted and/or displayed on a third-party device as described below, inputs to which may be processed as described in further detail below. Alternatively or additionally, biological extraction may be stored in and/or retrieved from a user database 124. User database 124 may include any data structure for ordered storage and retrieval of data, which may be implemented as a hardware or software module. A user database 124 may be implemented, without limitation, as a relational database, a key-value retrieval datastore such as a NOSQL database, or any other format or structure for use as a datastore that a person skilled in the art would recognize as suitable upon review of the entirety of this disclosure. A user database 124 may include a plurality of data entries and/or records corresponding to user tests as described above. Data entries in a user database 124 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a user database 124 may reflect categories, cohorts, and/or populations of data consistently with this disclosure. User database 124 may be located in memory of computing device 104 and/or on another device in and/or in communication with system 100.

With continued reference to FIG. 1, and as noted above, retrieval of biological extract may be performed multiple sequential and/or concurrent times, and any process using biological extract as described below may be performed multiple sequential and/or concurrent times; likewise, biological extract may include multiple elements of physiological data, which may be used in combination for any determination and/or other processes as described below.

With continued reference to FIG. 1, computing device 104 is configured to determine a nutritional output 128 utilizing an equivalency request 108, at least an element of user data 116, a user metabolic state, and at least a machine-learning process. A “nutritional output,” as used in this disclosure, is data identifying one or more meal possibilities for a user, customized around the user's personal nutritional needs. A “meal,” as used in this disclosure, is any eating occasion that takes place during the day. A meal may occur at a home, in a restaurant, at a cafeteria, at a workplace, and the like. For example, a meal may include breakfast, brunch, lunch, tea, dinner, supper, a combination meal, a snack, a kid's meal, and the like. A meal may consist of one or more elements. An ingredient may include any substance consumed to provide nutritional support for a user. An ingredient may contain essential nutrients such as carbohydrates, fats, proteins, vitamins, or minerals. An ingredient may be sourced from plants, animals, chemically altered substances inorganic substances and the like. For example, an ingredient may be sourced from one or more plants and may include corn, wheat, rice, beans, and/or nuts. In yet another non-limiting example, an ingredient may be sourced from one or more animals such as chicken, fish, beef, lamb, milk, dairy products, honey, eggs, and the like.

With continued reference to FIG. 1, a nutritional output 128 may identify one or more meal possibilities, that may be available for a user to consume. A meal possibility may identify one or more suggested meals that may be compatible and/or optimized around a user's unique nutritional needs. A meal possibility may identify a meal that a user can prepare at home, such as an acai bowl that a user can make for breakfast. A meal possibility may identify a meal that a user can order from a meal provider. A “meal provider,” as used in this disclosure, is any participant involved in the preparation and/or assembly of a meal possibility. A provider may include a restaurant such as a local privately owned restaurant or a chain restaurant that may be located at multiple locations. A provider may include a company that prepares pre-packaged meals. A provider may include a grocery store that prepares meals and may include a restaurant located within a grocery store. A provider may include a chef or cook who prepares meals at home or in a private commercialized kitchen. A provider may include a chef or cook who prepares meals in a school, kitchen, or space that the chef rents out.

With continued reference to FIG. 1, computing device 104 may identify a meal possibility based on a user's geolocation. A user's geolocation may identify a real-world geographical location of a user. A user's geolocation may be obtained from a radar source, a remote device 120 operated by a user such as a mobile phone, and/or an internet connected device location. A user geolocation 132 may include a global positioning system (GPS) of a user. A user geolocation 132 may include geographic coordinates that may specify the latitude and longitude of a particular location where a user is located.

With continued reference to FIG. 1, computing device 104 may adjust a nutritional output 128 based on the availability of certain elements contained within a nutritional output 128 based on the seasonal time of year, the availability of certain elements, and the geographic location of the user. Computing device 104 may evaluate a nutritional output 128 to identify available elements. An “available element,” as used in this disclosure, is any ingredient that is available to be included in a nutritional output. An ingredient is available when it exists and can be included in a nutritional output. For example, an ingredient may be seasonally available, and only available to be purchased at different times of the year. In yet another non-limiting example, an ingredient may be locally available, such as when an ingredient is only geographically available at certain locations. An ingredient may be available when certain quality standards and/or preferences are met for foods and/or supplements. For example, an ingredient may need to be grown and/or produced under certain conditions and contain non-genetically modified organisms (GMO), gluten free, dairy free, certified organic, elimination of a preservative and the like. In yet another non-limiting example, a supplement may need to be free of fillers that may cause irritation, allergy, or harm such as a supplement that contains a filler of lactose or magnesium stearate. In yet another non-limiting example, a supplement may need to contain certain grade ingredients including medical grade supplements, cosmetic grade supplements, nutritional grade supplements, and/or agricultural grade supplements. Computing device 104 may evaluate a nutritional output 128 to identify seasonably available elements. For example, elements such as apples, pumpkin, squash, and cranberries may be readily available in New England in the fall and winter months but may be scarce in the middle of the summer. Computing device 104 may evaluate a nutritional output 128 to identify geographic availability of elements. In yet another non-limiting example, ono fish may be prevalent and available to consume in Hawaii but may not be available to consume in other areas of the country such as in New York or Pennsylvania. Computing device 104 may adjust a nutritional output 128 upon evaluating a nutritional output 128 to identify available elements. Adjusting a nutritional output 128 may include removing one or more elements, adding in one or more elements, substituting one or more elements and the like. Computing device 104 may consult nutritional database 136 to determine what elements may not be seasonably available and may need to be substituted. Nutritional database may be implemented as any data structure suitable for use as user database 124 as described above. Nutritional database may contain information pertaining to availability of elements contained within a nutritional output 128, such as geographical availability of elements, seasonal availability of elements, and the like. Nutritional database 136 may be updated in real time to contain accurate information as to availability of particular elements. Nutritional database may contain information regarding compatibility and/or ability to substitute one or more elements when an ingredient may not be available due to it being a seasonal product, or not being able to be acquired in a certain geographical location. For example, nutritional database 136 may suggest that while sweet potato may not be seasonally available for a user, there may be an abundance of kabocha squash, and as such kabocha squash may be substituted in lieu of sweet potato. In yet another non-limiting example, computing device 104 may consult nutritional database to determine if a certain grade supplement is available, such as a medical grade ascorbic acid supplement, or a certified gluten free multi-vitamin supplement.

With continued reference to FIG. 1, computing device 104 determines a nutritional output 128 utilizing a machine-learning process. A “machine learning process” is a process that automatedly uses a body of data known as “training data” and/or a “training set” to generate an algorithm that will be performed by a computing device 104 and/or module to produce outputs given data provided as inputs; this is in contrast to a non-machine learning software program where the commands to be executed are determined in advance by a user and written in a programming language. At least a machine-learning process 140 may be used by computing device 104 to generate a nutritional output 128 as described in further detail below.

Still referring to FIG. 1, computing device 104 may store at least a machine-learning process 140 in and/or select at least a machine-learning process 140 from a process database 144. Process database 144 may include any database suitable for use as a user database 124 as described above; process database 144 may, as a non-limiting example, relate each machine-learning process 140 to an entry in one or more indices, such as indices of machine-learning process 140 identifiers, indices and/or links to tables of user data, biological extraction data, and/or identifiers of one or more equivalency request 108. One or more user textual entries may be mapped by a language processing module, model, and/or process to data used as index entries, which may then be used to form a query, which may combine such entries with user selections, to retrieve at least a machine-learning process 140.

With continued reference to FIG. 1, computing device 104 may select at least a machine-learning process 140 utilizing a specified individualized level. In an embodiment, one or more machine-learning process 140es may be stored within process database 144 and organized by specified individualized level. For instance and without limitation, computing device 104 may receive a user specified individualized level 112 that indicates the user would prefer a high level of individualized regarding the user's nutritional output 128. In such an instance, computing device 104 may select a machine-learning process 140 from within process database 144 intended for and queried to a high level of individualized. In yet another non-limiting example, computing device 104 may receive a user specified individualized level 112 that indicates the user would prefer a low level of individualized, and as such computing device 104 may select a machine-learning process 140 created for a low level of individualized.

With continued reference to FIG. 1, computing device 104 is configured to generate a nutritional output 128 utilizing a selected machine-learning process 140. Training data,” as used in this disclosure, is data containing correlations that a machine-learning process 140 may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning process 140es as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by computing device 104 may correlate any input data as described in this disclosure to any output data as described in this disclosure.

Still referring to FIG. 1, selection of at least a machine-learning process 140 may include selection of a machine-learning model, a training data set to be used in a machine-learning algorithm and/or to produce a machine-learning model, and/or a machine-learning algorithm such as lazy-learning and/or model production, or the like. Computing device 104 may be designed and configured to create a machine-learning model using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 1, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 1, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data.

Continuing to refer to FIG. 1, machine-learning algorithms may include supervised machine-learning algorithms. Supervised machine learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include elements of physiological data as described above as inputs, nutritional output 128 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data. Persons skilled in the art, upon reviewing the entirety of this disclosure, will be aware of various possible variations of supervised machine learning algorithms that may be used to determine relation between inputs and outputs. Supervised machine-learning process 140es may include classification algorithms as defined above.

Still referring to FIG. 1, machine learning process 140es may include unsupervised processes. An unsupervised machine-learning process 140, as used herein, is a process that derives inferences in datasets without regard to labels; as a result, an unsupervised machine-learning process 140 may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

With continued reference to FIG. 1, machine-learning process 140es as described in this disclosure may be used to generate machine-learning models. A machine-learning model, as used herein, is a mathematical representation of a relationship between inputs and outputs, as generated using any machine-learning process 140 including without limitation any process as described above, and stored in memory; an input is submitted to a machine-learning model once created, which generates an output based on the relationship that was derived. For instance, and without limitation, a linear regression model, generated using a linear regression algorithm, may compute a linear combination of input data using coefficients derived during machine-learning process 140es to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 1, at least a machine-learning process 140 may include a lazy-learning process and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data. Heuristic may include selecting some number of highest-ranking associations and/or training data elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

With continued reference to FIG. 1, computing device 104 is configured to calculate an optimization value 148 utilizing a nutritional output 128. An “optimization value,” as used in this disclosure, is any numerical and/or character data reflecting a budget or total dollar amount that it will cost the user to optimize nutrition for the user based on the nutritional output 128 and the user specified individualized level 112. An optimization value 148 may indicate how much money it will cost for a user's custom food supply. A food supply may include any quantity of food that may be consumed by a user over a specified period of time. For instance and without limitation, an optimization value 148 may specify that it will cost a user $100 each week for the user's entire food supply to be highly optimized around the user's own nutritional needs. In yet another non-limiting example, an optimization value 148 may specify that it will cost a user $75 each week for the user's entire food supply to be moderately optimized around the user's own nutritional needs.

With continued reference to FIG. 1, computing device 104 is configured to receive from a remote device 120, a maximum user optimization value 152. A “maximum user optimization value,” as used in this disclosure, is the maximum budget or total dollar amount that a user is willing to spend to optimize nutrition for the user based on the nutritional output 128 and the user specified individualized level 112. For example, a maximum user optimization value 152 may specify that a user is willing to spend no more than $60 per week on the user's food supply. In yet another non-limiting example, a maximum user optimization value 152 may specify that a user has no maximum amount of money that the user is willing to spend each week on food. Computing device 104 compares an optimization value 148 to a maximum user optimization value 152. In an embodiment, computing device 104 may compare an optimization value 148 to a maximum user optimization value 152 to ensure that an optimization value 148 does not exceed a maximum user optimization value 152. For example, computing device 104 may compare an optimization value 148 of $45 to a maximum user optimization value 152 of $75 to ensure that the optimization value 148 of $45 does not exceed the maximum user optimization value 152 of $75. Computing device 104 may minimize an optimization value 148 by substituting elements or recommending one or more less expensive elements contained within a nutritional output 128. Minimizing an optimization value 148 may include substituting one or more elements, choosing less expensive elements, removing one or more elements, and the like, to ensure that an optimization value 148 does not exceed a maximum user optimization value 152. In an embodiment, computing device 104 may minimize an optimization value 148 by consulting nutritional database 136. Nutritional database may contain one or more entries describing cost of one or more elements. For example, nutritional database 136 may describe the price per serving of an ingredient when purchased in a particular geographic location. For instance, nutritional database may reflect that a six ounce piece of lamb costs $12 per serving, while a six ounce piece of tofu costs $4 per serving.

With continued reference to FIG. 1, computing device 104 may subtract an optimization value 148 from a maximum user optimization value 152 to calculate a surplus. A “surplus,” as used in this disclosure, is any monetary value calculated when an optimization value 148 is subtracted from a maximum user optimization value 152. Computing device 104 may utilize a surplus to suggest one or more lifestyle outputs. A “lifestyle output,” as used in this disclosure, is any activity that has a positive impact on a user's life and will help promote health and longevity. A lifestyle output may include a fitness suggestion such as an exercise program or class that may be of benefit for a user. For example, a lifestyle output may suggest that a user should utilize a surplus to join a gym and engage in exercise for at least thirty minutes three times each week. A lifestyle output may include a spiritual suggestion, such as a meditation subscription that a user can purchase to practice a meditation sequence for 15 minutes each night before bed. One or more lifestyle outputs may be stored within nutritional database 136. Computing device 104 may retrieve one or more lifestyle output from nutritional database 136 to generate a suggested lifestyle output.

With continued reference to FIG. 1, an optimization value 148 may be adjusted based on one or more user inputs. Computing device 104 may receive a user input utilizing any network methodology as described herein. In an embodiment, a user input may contain a specified period of time that a user seeks to have an optimization value 148 calculated for. For example, a user may generate a user input that requests an optimization value 148 be calculated for a user for the upcoming two weeks, as the user will not be traveling during that time period and will be at home and may be able to prepare one or more meal possibilities at home. In yet another non-limiting example, an optimization value 148 may be calculated for a user for a specified period of time such as three days because the user is traveling in a city for three days. One or more user inputs may specify certain geographical areas where the user is located, how often the user likes to prepare meals at home versus eating out, ingredient preferences and the like. For example, a user input may specify that a user does not eat any animal containing products due to ethical reasons or that a user does not consume eggs because the user has an aversion to eggs.

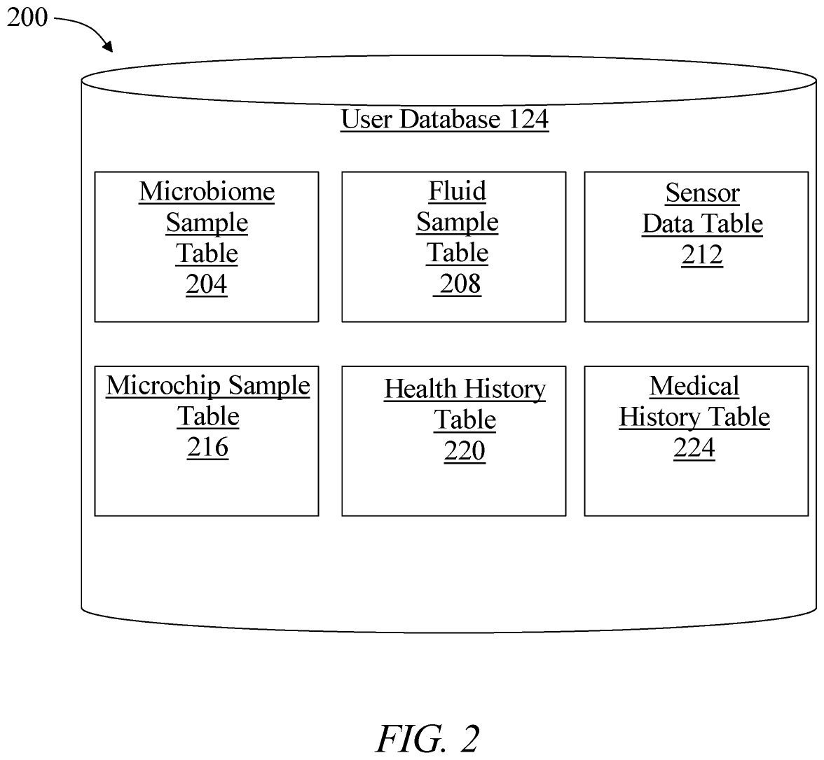

Referring now to FIG. 2, an exemplary embodiment 200 of user database 124 is illustrated. User database 124 may be implemented as any data structure as described above in more detail in reference to FIG. 1. One or more tables contained within user database 124 may include microbiome sample table 204; microbiome sample table 204 may include one or more biological extraction relating to the microbiome. For instance and without limitation, microbiome sample table 204 may include a physically extracted sample such as a stool sample analyzed for the presence of pathogenic species such as parasites and anaerobes. One or more tables contained within user database 124 may include fluid sample table 208; fluid sample table 208 may include one or more biological extraction containing fluid samples. For instance and without limitation, fluid sample table 208 may include a urine sample analyzed for the presence or absence of glucose. One or more tables contained within user database 124 may include sensor data table 212; sensor data table 212 may include one or more biological extraction containing sensor measurements. For instance and without limitation, sensor data table 212 may include heart rate, blood pressure, and glucose readings. One or more tables contained within user database 124 may include microchip sample table 216; microchip sample table 216 may include one or more biological extraction obtained from a microchip. For instance and without limitation, microchip sample table 216 may include an intracellular nutrient level obtained from a microchip embedded under a user's skin. One or more tables contained within user database 124 may include health history table 220; health history table may include one or more elements of data pertaining to a user's health history. For instance and without limitation, health history table 220 may contain a user's self-reported previous diagnosis of hypertension. One or more tables contained within user database 124 may include medical history table 224; medical history table 224 may include one or more elements of data pertaining to a user's medical history. For instance and without limitation, medical history table 224 may include a user's previous surgical history including a previous ankle bone surgery.

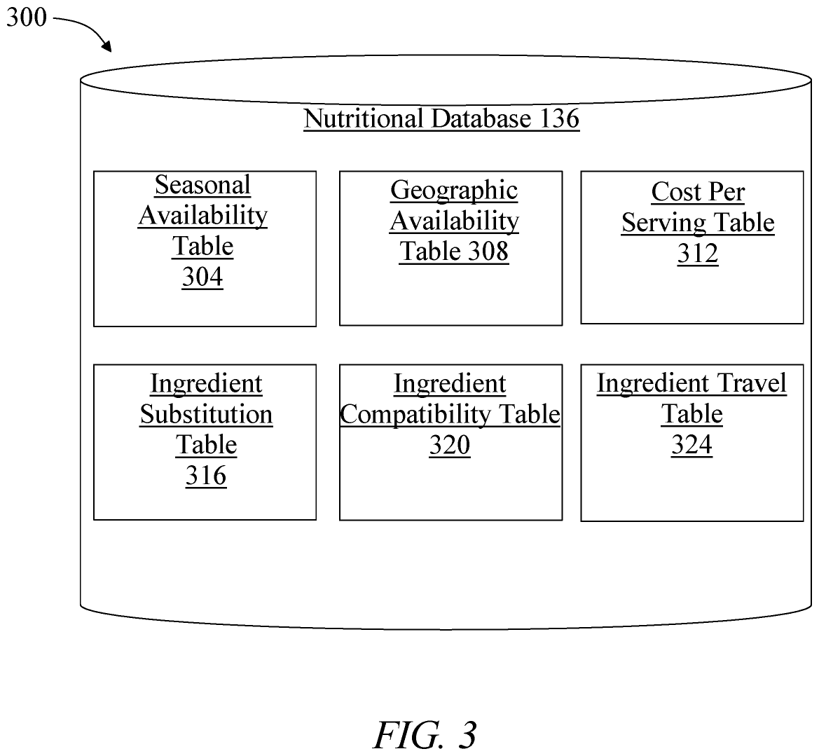

Referring now to FIG. 3, an exemplary embodiment 300 of nutritional database 136 is illustrated. Nutritional database 136 may be implemented as any data structure as described above in more detail in reference to FIG. 1. Nutritional database 136 may be implemented as any data structure suitable for use as described above in more detail in reference to FIG. 1. One or more tables contained within nutritional database 136 may include seasonal availability table 304; seasonal availability table 304 may include information describing the seasonal availability of one or more elements. For instance and without limitation, seasonal availability table 304 may describe what elements are available in abundance in fall, winter, spring, and summer. One or more tables contained within nutritional database 136 may include geographic availability table 308; geographic availability table 308 may include information describing the geographic availability of one or more elements. For instance and without limitation, geographic availability table 308 may describe what elements are available in Honolulu, Hi. One or more tables contained within nutritional database 136 may include cost per serving table 312; cost per serving table 312 may include information describing the cost per serving of one or more elements. For instance and without limitation, cost per serving table 312 may include information describing the cost per serving of filet mignon. One or more tables contained within nutritional database 136 may include ingredient substitution table 316; ingredient substitution table 316 may include information describing elements that may be substituted based on flavor, taste, and/or ability to be incorporated into a particular meal possibility. For instance and without limitation, ingredient substitution table 316 may include information describing what elements can be substituted for a white potato. One or more tables contained within nutritional database 136 may include ingredient compatibility table 320; ingredient compatibility table 320 may include information describing the compatibility of elements based on a particular nutritional need. For instance and without limitation, ingredient compatibility table 320 may describe what ingredient is compatible for a user with a nutritional need such as low iron levels. One or more tables contained within nutritional database 136 may include ingredient travel table 324; ingredient travel table 324 may include information describing how far a particular ingredient traveled from where it was grown, and where it was sourced from. For instance and without limitation, ingredient travel table 324 may describe where an apple was grown and produced.

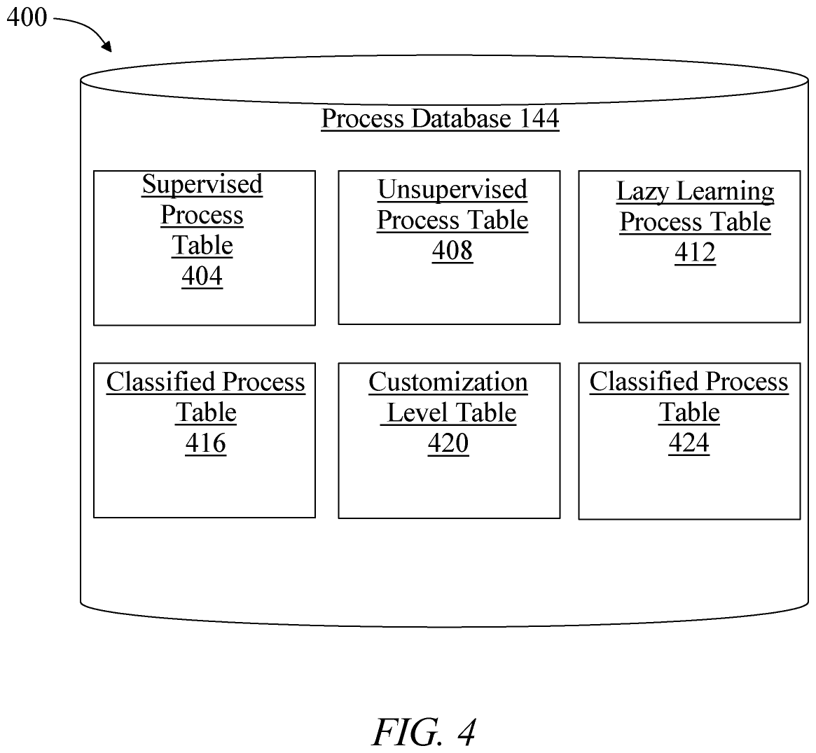

Referring now to FIG. 4, an exemplary embodiment 400 of process database 144 is illustrated. Process database 144 may be implemented as any data structure as described above in more detail in reference to FIG. 1. One or more tables contained within process database 144 may include supervised process table 404; supervised process table 404 may include one or more supervised processes. One or more tables contained within process database 144 may include unsupervised process table 408; unsupervised process table 408 may include one or more unsupervised processes. One or more tables contained within process database 144 may include lazy learning process table 412; lazy learning process table 412 may include one or more lazy learning processes. One or more tables contained within process database 144 may include classified process table 416; classified process table 416 may include one or more classified processes. One or more tables contained within process database 144 may include individualized level table 420; individualized level table 420 may include one or more processes classified by individualized level. One or more tables contained within process database 144 may include classified process table 424; classified process table 424 may include one or more classified processes.

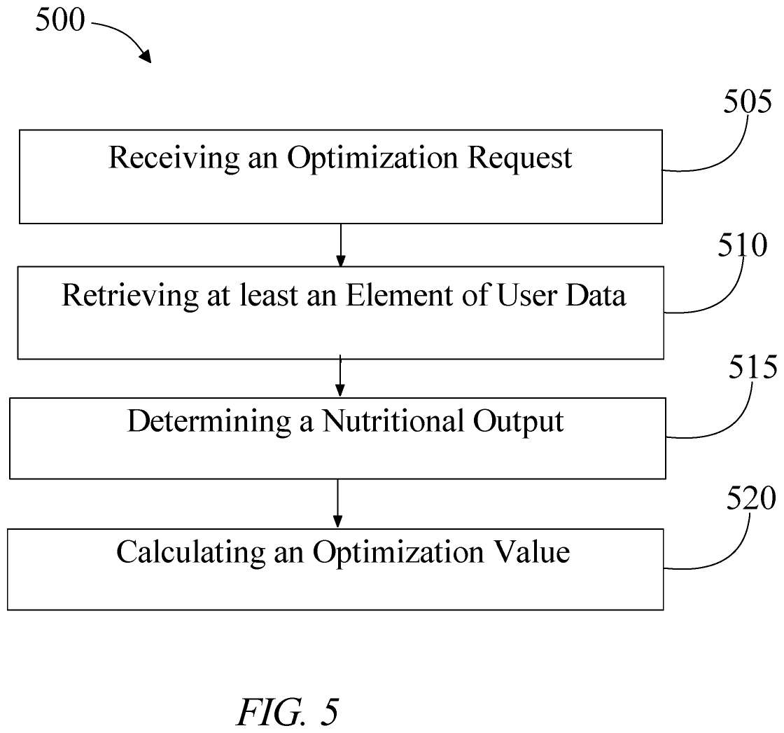

Referring now to FIG. 5, an exemplary embodiment 500 of an artificial intelligence method of multi-factor selection process is illustrated. At step 505, a computing device 104 receives an equivalency request 108. An equivalency request 108 includes any of the equivalency request 108 as described above in more detail in reference to FIG. 1. An equivalency request 108 indicates how personalized a user desires to have nutrition and/or a food supply optimized to the user's body. An equivalency request 108 contains a user specified individualized level 112. An individualized level may indicate how customized to the user's own nutritional needs the user seeks to have the user's food supply be generated for the user. An equivalency request 108 may be generated on a continuum, where there may be a scaled response indicating how personalized and/or customized a user desires a meal personalized. For example, an equivalency request 108 may contain a request for a user to have the user's food supply highly optimized to the user's nutritional needs. In yet another non-limiting example, an equivalency request 108 may contain a request for a user to not have the user's food supply highly optimized to the user's nutritional needs, and instead to only have the user's food supply to be generated for the user.

With continued reference to FIG. 5, at step 510 a computing device retrieves at least an element of user data 116 and determines a user metabolic state utilizing at least a retrieved element of user data and a classification algorithm. At least an element of user data 116 includes any of the at least an element of user data 116 as described above in more detail in reference to FIG. 1. At least an element of user data 116 may include a user reported element of user data 116. For example, at least an element of user data 116 may contain a user self-reported description of a user's previously diagnosed illnesses. In an embodiment, at least an element of user data 116 may contain a list of one or more drug allergies that the user was previously diagnosed with. At least an element of user data 116 may contain any information pertaining to a user's previous medical and/or health history reported by a user. In an embodiment, at least an element of user data 116 may contain a biological extraction. A biological extraction may include any of the biological extractions as described above in more detail in reference to FIG. 1. For example, a biological extraction may contain a blood test analyzed for intracellular nutrient levels of various nutrients including but not limited to Vitamin A, Vitamin D, Vitamin C, Vitamin E, Vitamin B1 and the like. In yet another non-limiting example, a biological extraction may contain a stool sample analyzed for various gut microbe strains. One or more elements of user data may be stored within user database 124, as described above in more detail in reference to FIGS. 1-4. In an embodiment, computing device 104 may utilized an equivalency request 108 to select at least an element of user data 116 relating to an equivalency request 108. For instance and without limitation, an equivalency request 108 that contains an indication that a user does not want their food supply highly optimized, and instead would prefer to have a food supply that is very low in being optimized around the user's nutritional needs, may lead to computing device 104 to select any element of user data 116 stored within user database 124. In yet another non-limiting example, an equivalency request 108 that contains an indication that a user does want their food supply highly optimized may lead to computing device 104 to select a plurality of elements of user data stored within user database 124. In such an instance, computing device 104 may select a variety of elements of user data that may relate to different aspects of health, such as an element of user data 116 that relates to the microbiome, an element of user data 116 that relates to the gut wall, an element of user data 116 that relates to genetics, an element of user data 116 that relates to toxicity, an element of user data 116 that relates to epigenetics and the like. In an embodiment, computing device 104 may select one or more elements of user data based on input from experts. Expert input as to which elements of user data need to be selected may be stored within a database located within system 100, such as in memory. Experts in the field of health, functional medicine, and the like may suggest which elements of user data may be necessary to optimize nutritional levels based on various specified individualized levels. For example, an individualized level that requests only low levels of optimization may require any element of a user's health history to generate a nutritional output 128, while an individualized level that requests high levels of optimization may require at least two different elements of user data such as a vitamin blood panel and a stool microbiome sample.

With continued reference to FIG. 5, computing device 104 generates a classification algorithm, wherein the classification algorithm utilizes at least an element of user data as an input and outputs a user metabolic state. A user metabolic state includes any of the user metabolic states as described above in more detail in reference to FIG. 1. Classification algorithm includes any of the classification algorithms as described above in more detail in reference to FIG. 1. Classification algorithm may include an algorithm such as fisher's linear discriminant, or a support vector machine. One or more classification algorithms may be selected and/or utilized based on expert input, including any of the expert input as described herein. Computing device 104 identifies using a classification algorithm and the at least an element of user data a user metabolic state. A user metabolic state may indicate one or more markers of user metabolism and/or user physical activity. For example, a user metabolic state may indicate that a user is a fast metabolizer of food elements and the user is engaged in vigorous physical activity multiple times each week. In yet another non-limiting example, a user metabolic state may indicate that a user is an average metabolizer of food elements, and the user is currently not engaged in any physical activity. A user metabolic state may be obtained utilizing at least an element of user data that may contain one or more readings from a computer connected to a sensor. A reading may indicate one or more bio-physical signals such as a user's heart rate, or the respiration rate of a user at rest. A reading may indicate a user's heart rate variability or how well hydrated the epidermis and dermis of the user's skin are.

With continued reference to FIG. 5, at step 515, a computing device 104 determines a nutritional output 128 utilizing an equivalency request 108, at least an element of user data 116, a user metabolic state, and at least a machine-learning process. A nutritional output 128 includes any of the nutritional output 128 as described above in more detail in reference to FIG. 1. A nutritional output 128, identifies one or more meal possibilities for a user, customized around the user's personal nutritional needs. A meal possibility includes a suggestion as to meals that a user could consume that would be customized around the user's personal nutritional needs and optimize the user's nutrition. For example, a nutritional output 128 may suggest a breakfast for a user who seeks to have meals to highly customized for the user's nutritional needs, to contain chia seed pudding made with organic almond milk, hemp seeds, and mango, because the mango has lots of Vitamin C, which will help boost the user's immune system during winter months when the user may be susceptible to disease. A meal possibility may identify one or more meals that a user could order from a restaurant located within a certain geographical distance of the user. For example, a meal possibility may identify a meal that a user can order from a restaurant for lunch that is next door to the user's office building that will optimize the user's nutritional needs. In yet another non-limiting example, a meal possibility may identify a meal that a user can order from any meal provider as described above in more detail in reference to FIG. 1.

With continued reference to FIG. 5, computing device 104 determines a nutritional output 128 utilizing a machine-learning process 140. A machine-learning process 140 may include any of the machine-learning process 140es as described above in more detail in reference to FIGS. 1-4. One or more machine-learning process 140es may be stored in process database 144 as described above in more detail. Computing device 104 may select at least a machine-learning process 140 from process database 144. In an embodiment, computing device 104 may select at least a machine-learning process 140 utilizing a specified individualized level. One or more machine-learning process 140es may be stored and queried within process database 144 based on individualized level. For example, an individualized level that indicates high individualized may be best suited for a machine-learning process 140 that includes supervised machine-learning process 140, and as such computing device 104 may select a supervised machine-learning process 140 when a user seeks high individualized. In yet another non-limiting example, an individualized level that indicates low individualized may be best suited for a machine-learning process 140 that includes a hierarchical clustering process, and as such computing device 104 may select a hierarchical clustering machine-learning process 140. Computing device 104 generates a nutritional output 128 utilizing a selected machine-learning process 140, at least an element of user data 116, and an equivalency request 108. Computing device 104 may generate a nutritional output 128 utilizing a selected machine-learning process 140 utilizing any of the methods as described above in more detail in reference to FIGS. 1-4.

With continued reference to FIG. 5, computing device 104 calculates an optimization value 148 utilizing a nutritional output 128. An optimization value 148 includes any of the optimization value 148 as described above in more detail in reference to FIG. 1. An optimization value 148 is any numerical and/or character data reflecting a budget or total dollar amount that it will cost the user to optimize nutrition for the user based on the nutritional output 128 and the user specified individualized level 112. In an embodiment, an optimization value 148 may contain a total dollar amount that it will cost the user to optimize nutrition for the user. For example, an optimization value 148 may reflect that it may cost a user $110 each week to highly optimize nutrition around the user's personal nutrition needs. In an embodiment, an optimization value 148 may be calculated for a specified period of time. For example, a user may generate an input stored within user database 124 that contains a request to receive an optimization value 148 calculated for a two week period or for a six month period.

With continued reference to FIG. 5, computing device 104 may evaluate a nutritional output 128 based on available elements. Available elements may include any of the available elements as described above in more detail in reference to FIG. 1. For example, computing device 104 may evaluate a nutritional output 128 to determine if an ingredient is seasonally available or if an ingredient is geographically available. For example, an ingredient such as hazelnut may be prevalent and easy to incorporate in a meal possibility in the Pacific Northwest but may be difficult to incorporate in a meal possibility in the South. In yet another non-limiting example, it may be difficult to locate fresh mango and fresh papaya in Alaska, but it may be easy to locate such elements in Hawaii. Computing device 104 may evaluate a nutritional output 128 to identify available elements such as by consulting nutritional database. Nutritional database 136 may contain an accurate and/or real time updated list of elements that may be seasonally and/or geographically available or able to be obtained. Computing device 104 may adjust a nutritional output 128 utilizing available elements. For example, computing device 104 may adjust a nutritional output that includes an ingredient such as a macoun apple that is only available in October in New England, and instead substitute an ingredient such as a Macintosh apple, that is available year round in New England. Computing device 104 may consult nutritional database 136 to determine which elements can be substituted as well as which elements that can be substituted will also continue to optimize a user's nutrition. For example, computing device 104 may consult nutritional database to determine if a substitute, such as using butternut squash in lieu of kabocha squash will still continue to optimize a user's nutritional status. In yet another non-limiting example, computing device 104 may consult nutritional database 136 to determine if certain ingredients grown under certain conditions are available. For example, computing device 104 may determine if an ingredient such as lettuce that is grown without the use of pesticides is available. In yet another non-limiting example, computing device 104 may consult nutritional database 136 to evaluate if a medical grade cranberry extract supplement is available to be included in a nutritional output. Computing device 104 adjusts a nutritional output 128 if an ingredient is not available. For instance and without limitation, computing device 104 may determine that grass-fed free range ground beef is not available to be included in a nutritional output, and as such computing device 104 adjusts the nutritional output to include grass-fed free range ground lamb instead. In yet another non-limiting example, computing device 104 may determine that a food grade milk thistle supplement is not available, and instead computing device 104 may consult nutritional database 136 to substitute a medical grade milk thistle supplement instead.

With continued reference to FIG. 5, computing device 104 may adjust a nutritional output 128 based on user input regarding a nutritional output 128. Computing device 104 may receive from a remote device 120, a maximum user optimization value 152. Maximum user optimization value 152 includes any of the maximum user optimization value 152 as described above in more detail in reference to FIGS. 1-4. Maximum user optimization value 152 may contain a specified amount of money that a user is willing to spend on a user's meals each month. For example, a maximum user optimization value 152 may specify that a user is willing to spend up to $150 each week on all expenses relating to meals. In yet another non-limiting example, a maximum user optimization value 152 may specify that a user has an unlimited budget and has no limit on the amount of money that the user will spend each week on all expenses relating to meals. Computing device 104 may compare an output optimization value 148 to a maximum user optimization value 152 and minimize the output optimization value 148. Minimizing the output optimization value 148 may include select less expensive elements and/or suggesting less costly meal possibilities so that an optimization value 148 does not exceed a user's maximum user optimization value 152. Computing device 104 may minimize an optimization value 148 by consulting nutritional database 136. For example, computing device 104 may consult nutritional database 136 to determine a cost per serving of a particular ingredient, to know which elements can be substituted. For example, computing device 104 minimize an optimization value 148 by consulting nutritional database 136 to determine what ingredient can be substituted for fresh pineapple that will cost less per serving than pineapple and will also be compatible with a user's body and provide sufficient nutrition to a user. Computing device 104 may subtract an optimization value 148 from a maximum user optimization value 152 to calculate a surplus. A surplus includes any of the surplus as described above in more detail in reference to FIG. 1. Computing device 104 utilizes a surplus to suggest a lifestyle output. A lifestyle output includes any of the lifestyle outputs as described above in more detail in reference to FIG. 1. A lifestyle output identifies any activity that has a positive impact on a user's life and will help promote health and longevity. For instance and without limitation, a lifestyle output may suggest a hot yoga class twice a week that a user can engage in to help reduce the user's stress and anxiety. A lifestyle output may suggest a meditation class that a user can engage in to develop a meditation practice. A lifestyle output may suggest an activity that a user can participate in, such as a knitting club to help the user develop a social network. A lifestyle output may suggest an organization that a user can consider donating money and volunteer work to, such as a local church organization.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

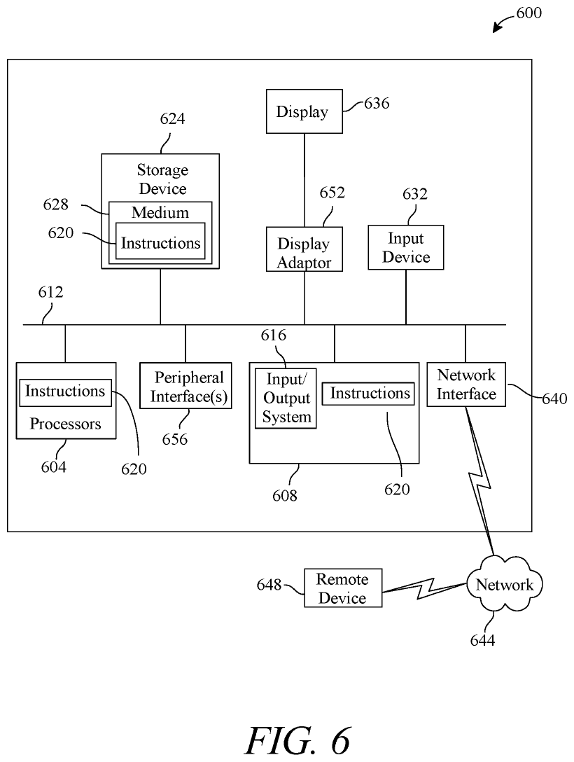

FIG. 6 shows a diagrammatic representation of one embodiment of a computing device in the exemplary form of a computer system 600 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 600 includes a processor 604 and a memory 608 that communicate with each other, and with other components, via a bus 612. Bus 612 may include any of several types of bus structures including, but not limited to, a memory bus, a memory controller, a peripheral bus, a local bus, and any combinations thereof, using any of a variety of bus architectures.

Memory 608 may include various components (e.g., machine-readable media) including, but not limited to, a random access memory component, a read only component, and any combinations thereof. In one example, a basic input/output system 616 (BIOS), including basic routines that help to transfer information between elements within computer system 600, such as during start-up, may be stored in memory 608. Memory 608 may also include (e.g., stored on one or more machine-readable media) instructions (e.g., software) 620 embodying any one or more of the aspects and/or methodologies of the present disclosure. In another example, memory 608 may further include any number of program modules including, but not limited to, an operating system, one or more application programs, other program modules, program data, and any combinations thereof.

Computer system 600 may also include a storage device 624. Examples of a storage device (e.g., storage device 624) include, but are not limited to, a hard disk drive, a magnetic disk drive, an optical disc drive in combination with an optical medium, a solid-state memory device, and any combinations thereof. Storage device 624 may be connected to bus 612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 624 (or one or more components thereof) may be removably interfaced with computer system 600 (e.g., via an external port connector (not shown)). Particularly, storage device 624 and an associated machine-readable medium 628 may provide nonvolatile and/or volatile storage of machine-readable instructions, data structures, program modules, and/or other data for computer system 600. In one example, software 620 may reside, completely or partially, within machine-readable medium 628. In another example, software 620 may reside, completely or partially, within processor 604.

Computer system 600 may also include an input device 632. In one example, a user of computer system 600 may enter commands and/or other information into computer system 600 via input device 632. Examples of an input device 632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 632 may be interfaced to bus 612 via any of a variety of interfaces (not shown) including, but not limited to, a serial interface, a parallel interface, a game port, a USB interface, a FIREWIRE interface, a direct interface to bus 612, and any combinations thereof. Input device 632 may include a touch screen interface that may be a part of or separate from display 636, discussed further below. Input device 632 may be utilized as a user selection device for selecting one or more graphical representations in a graphical interface as described above.

A user may also input commands and/or other information to computer system 600 via storage device 624 (e.g., a removable disk drive, a flash drive, etc.) and/or network interface device 640. A network interface device, such as network interface device 640, may be utilized for connecting computer system 600 to one or more of a variety of networks, such as network 644, and one or more remote device 120 648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 644, may employ a wired and/or a wireless mode of communication. In general, any network topology may be used. Information (e.g., data, software 620, etc.) may be communicated to and/or from computer system 600 via network interface device 640.

Computer system 600 may further include a video display adapter 652 for communicating a displayable image to a display device, such as display device 636. Examples of a display device include, but are not limited to, a liquid crystal display (LCD), a cathode ray tube (CRT), a plasma display, a light emitting diode (LED) display, and any combinations thereof. Display adapter 652 and display device 636 may be utilized in combination with processor 604 to provide graphical representations of aspects of the present disclosure. In addition to a display device, computer system 600 may include one or more other peripheral output devices including, but not limited to, an audio speaker, a printer, and any combinations thereof. Such peripheral output devices may be connected to bus 612 via a peripheral interface 656. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An artificial intelligence system for multi-factor selection process, the system comprising a computing device, the computing device designed and configured to:
receive an equivalency request, wherein the equivalency request contains a user specified individualized level;
retrieve at least an element of user data and determine a user metabolic state utilizing the at least a retrieved element of user data and a classification algorithm;
determine a nutritional output utilizing the equivalency request, the at least an element of user data, the user metabolic state and at least a machine-learning process; and
calculate an optimization value utilizing the nutritional output.

2. The system of claim 1, wherein the computing device utilizes the equivalency request to select the at least an element of user data relating to the equivalency request.

3. The system of claim 1, wherein the at least an element of user data contains a user reported element of user data.

4. The system of claim 1, wherein the at least an element of user data contains a biological extraction.

5. The system of claim 1, wherein the computing device is further configured to:
generate, the classification algorithm, wherein the classification algorithm utilizes the at least an element of user data as an input and outputs a user metabolic state; and
identify, using the classification algorithm and the at least an element of user data a user metabolic state.

6. The system of claim 1, wherein the computing device is further configured to:
select the at least a machine-learning process utilizing the user specified individualized level.

7. The system of claim 1, wherein the computing device is further configured to:
assess the nutritional output to identify available elements; and
adjust the nutritional output utilizing available elements.

8. The system of claim 1, wherein the computing device is further configured to:
receive, from a remote device, a maximum user optimization value;
compare the optimization value to the maximum user optimization value; and
minimize the optimization value.

9. The system of claim 8, wherein the computing device is further configured to:
subtract the optimization value from the maximum user optimization value to calculate a surplus; and
utilize the surplus to suggest a lifestyle output.

10. The system of claim 1, wherein the computing device is further configured to calculate the optimization value for a specified period of time.

11. An artificial intelligence method of multi-factor selection process, the method comprising:
receiving, by a computing device, an equivalency request, wherein the equivalency request contains a user specified individualized level;
retrieving, by the computing device, at least an element of user data and determining a user metabolic state utilizing at least a retrieved element of user data and a classification algorithm;
determining, by the computing device, a nutritional output utilizing the equivalency request, the at least an element of user data, the user metabolic state, and at least a machine-learning process; and
calculating by the computing device an optimization value utilizing the nutritional output.

12. The method of claim 11, wherein retrieving the at least an element of user data, further comprises utilizing the equivalency request to select the at least an element of user data relating to the equivalency request.

13. The method of claim 11, wherein retrieving the at least an element of user data further comprises retrieving a user reported element of user data.

14. The method of claim 11, wherein retrieving the at least an element of user data further comprises retrieving a biological extraction.

15. The method of claim 11, wherein determining by the computing device the nutritional output further comprises:
generating the classification algorithm, wherein the classification algorithm utilizes the at least an element of user data as an input and outputs a user metabolic state; and
identifying, using the classification algorithm and the at least an element of user data, a user metabolic state.

16. The method of claim 11, wherein selecting the at least a machine-learning process further comprises selecting the at least a machine-learning process utilizing the user specified individualized level.

17. The method of claim 11, wherein calculating the optimization value further comprises:
assessing the nutritional output to identify available elements; and
adjusting the nutritional output utilizing available elements.

18. The method of claim 11, wherein calculating the optimization value further comprises:
receiving, from a remote device, a maximum user optimization value;
comparing the optimization value to the maximum user optimization value; and
minimizing the optimization value.

19. The method of claim 18 further comprising:
subtracting the optimization value from the maximum user optimization value to calculate a surplus; and
utilizing the surplus to suggest a lifestyle output.

20. The method of claim 11, wherein calculating the optimization value further comprises calculating the optimization value for a specified period of time.
